(19) United States
(12) Patent Application Publication
(10) Pub. No.: US 2014/0212877 A1
(43) Pub. Date: Jul. 31, 2014

(54) MAIZE LINKAGE DRAG AND GENOME ANALYSIS PROCESS

(71) Applicant: Agrigenetics, Inc., Indianapolis, IN (US)

(72) Inventors: Lindsay R. Manley, Indianapolis, IN (US); Joseph W. Spinks, Indianapolis, IN (US); Trisha Borowicz, Greenfield, IN (US); Roanne Pangborn, Indianapolis, IN (US)

(73) Assignee: AGRIGENETICS, INC., Indianapolis, IN (US)

(21) Appl. No.: 14/167,165

(22) Filed: Jan. 29, 2014

Related U.S. Application Data
(60) Provisional application No. 61/758,310, filed on Jan. 30, 2013.

(51) Int. Cl. C12Q 1/68

(52) U.S. Cl. CPC C12Q 1/6895
USPC 435/6.11

(57) ABSTRACT

This invention relates to a method for improving a maize linkage drag and genome analysis process. Some embodiments produce a significant cost and time reduction in plant breeding projects. Particular embodiments concern a method to determine one amount of linkage drag flanking a desired, introgressed trait and to determine a recurrent parent percentage performed at a same stage of plant growth. This disclosure also concerns selecting plants containing the desired, introgressed trait based on results of genome analysis.

This application claims a priority based on provisional application 61/758,310 which was filed in the U.S. Patent and Trademark Office on Jan. 30, 2013, the entire disclosure of which is hereby incorporated by reference.

### BACKGROUND

Plant breeding allows for the introduction of desired traits into specific varieties of plants. This happens at the genetic level with the introduction of specific genes or specific alleles. When a gene or allele is transfer from a donor parent, not just the gene is transferred. Rather upstream and downstream chromosomal regions are transferred as well, which is known as “linkage drag”. This can be detrimental since undesirable DNA that can negatively affect crop performance may be linked to the target gene from the donor parent (Allard, Principles of Plant Breeding, 1999). Continued backcrossing can be performed to attempt to eliminate the adjacent DNA Improved processes are described to determine the linkage drag and recurrent parent percentage to determine plants comprising minimal to no linkage drag.

### SUMMARY OF INVENTION

Processes described herein are improved processes providing a streamlined linkage drag and genomic analysis. Embodiments as disclosed herein produce a significant cost and time reduction in plant breeding projects. After introgression of a desired gene or allele to produce progeny with a desired trait, genomes of progeny are analyzed to determine the amount of linkage drag.

An embodiment includes a method for analyzing plants which contain a desired, introgressed trait comprising analyzing a genome of a plant containing the desired, introgressed trait by determining at least one amount of linkage drag flanking the desired trait on a chromosome containing the desired trait in the genome of the plant; and determining a recurrent parent percentage present on all chromosomes in the genome of the plant, wherein both the determination of the linkage drag and the determination of recurrent parent percentage are performed at a same stage of plant growth. An embodiment further comprises selecting plants containing the desired, introgressed trait based on results of genome analysis.

In a preferred embodiment, a 13 step process is reduced to a 6 step process. An intermediate plate is created. DNA is then dispensed to PCR plates (e.g., 1536 well plate). The DNA is dried. After drying, a PCR cocktail is dispensed to the PCR plates. The PCR plates undergo thermal cycling (i.e., the PCR reaction). Once the thermal cycling is complete, the plates are then read by a fluorescent plate reader. This process can be conducted as an automated high throughput process.

### DETAILED DESCRIPTION

### Definitions

The term “crossing” refers to the fertilization of female plants (or gametes) by male plants (or gametes).

The terms “introgression”, “introgressed” and “introgressing” refer to both a natural and artificial process, and the resulting events, whereby genes of one species, variety or cultivar are moved into the genome of another species, variety or cultivar, by crossing those species. The process may optionally be completed by backcrossing to the recurrent parent. To achieve introgression of only a part of a chromosome of one plant into a chromosome of another plant, random portions of the genomes of both parental lines recombine during the cross due to the occurrence of crossing-over events in the production of gametes in the parent lines.

The term “recurrent parent” refers to the parent to which the first cross and successive backcrossed plants are crossed.

The term “single cross” is a cross between two genotypes, usually two genetically different inbred lines or synthetic lines.

The term “donor parent” refers to the parent from which one or a few genes are transferred to the recurrent parent in backcross breeding.

The term “elite inbred” or “elite genotype” is any plant line that has resulted from breeding and selection for superior agronomic performance.

The term “plant,” includes plants and plant parts including but not limited to plant cells and plant tissues such as leaves, stems, roots, flowers, pollen, and seeds. A plant can be, but is not limited to, maize, cotton, soybean, sorghum, rice, etc.

The term “allele” refers to any of one or more alternative form of a gene, all of which alleles relates to one trait or characteristic. In a diploid cell or organism, two alleles of a given gene occupy corresponding loci on a pair of homologous chromosomes.

“Polymerase chain reaction” or “PCR” refers to a procedure or technique in which minute amounts of nucleic acid are amplified as described in U.S. Pat. No. 4,683,195, issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify, inter alia, specific DNA sequences from total genomic DNA. See generally Mullis et al., Cold Spring Harbor Symp. Quant. Biol., 51:263 (1987); Erlich, ed., PCR Technology, (Stockton Press, NY, 1989).

The term “oligonucleotide” refers to a single-stranded nucleic acid including at least between two and about 100 natural or modified nucleotides or a mixture thereof. The oligonucleotide can be derived from a natural nucleic acid or produced by chemical or enzymatic synthesis.

The term “probe” refers to an oligonucleotide that hybridizes to a target sequence. In the TaqMan® or TaqMan®-style assay procedure, the probe hybridizes to a portion of the target situated between the annealing site of the two primers. A probe can further include a detectable label, e.g., a fluorophore (TexasRed®, Fluorescein isothiocyanate, etc.,). The detectable label can be covalently attached directly to the probe oligonucleotide, e.g., located at the probe's 5′ end or at the probe's 3′ end. A probe including a fluorophore may also further include a quencher, e.g., Black Hole Quencher™, Iowa Black™, etc. A probe includes about eight nucleotides, about ten nucleotides, about fifteen nucleotides, about twenty nucleotides, about thirty nucleotides, about forty nucleotides, or about fifty nucleotides. In some embodiments, a probe includes from about eight nucleotides to about fifteen nucleotides.

The term “quenching” refers to a decrease in fluorescence of a fluorescent detectable label caused by energy transfer associated with a quencher moiety, regardless of the mechanism.

The term “reaction mixture” or “PCR reaction mixture” or “RT-PCR reaction mixture” or “master mix” or “master mixture” refers to an aqueous solution of constituents in a PCR or RT-PCR reaction that can be constant across different reactions. An exemplary RT-PCR reaction mixture includes buffer, a mixture of deoxyribonucleoside triphosphates, reverse transcriptase, primers, probes, and DNA polymerase. Generally, template RNA or DNA is the variable in a PCR or RT-PCR reaction.

The term “nucleic acid molecule” (or “nucleic acid” or “polynucleotide”) refers to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. The term may refer to a molecule of RNA or DNA of indeterminate length. The term includes single- and double-stranded forms of DNA. A nucleic acid molecule may include either or both naturally-occurring and modified nucleotides linked together by naturally occurring and/or non-naturally occurring nucleotide linkages.

The term “oligonucleotide” refers to a short nucleic acid polymer. Oligonucleotides may be formed by cleavage of longer nucleic acid segments, or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in PCR, a technique for the amplification of small DNA sequences. In PCR, the oligonucleotide is typically referred to as a “primer,” which allows a DNA polymerase to extend the oligonucleotide and replicate the complementary strand.

The term “reaction mixture” or “PCR reaction mixture” or “PCR cocktail (CKTL)” refers to an aqueous solution of constituents in a PCR reaction that can be constant across different reactions. An exemplary PCR reaction mixture includes buffer, a mixture of deoxyribonucleoside triphosphates, reverse transcriptase, primers, probes, and DNA polymerase. Generally, template DNA is the sole variable in a PCR reaction.

### Introgression

Plant breeding introduces gene(s) or a specific allele from a donor parent to provide or improve a particular trait. Introgression of a desirable trait in plants may be facilitated by repeated backcrossing. Preferably, a gene or allele is introduced via introgression. During the cross, not just the specific gene or allele is present in the progeny. There is also adjacent chromosomal DNA (upstream and/or downstream of the genetic locus) that is also introduced to the progeny. Preferably, the amount of adjacent DNA that is present in the progeny is to be minimized The adjacent DNA that transfers can have a deleterious effect. After introgressing the desired gene or allele, the amount of adjacent DNA that transfers to the progeny can be measured (e.g., RFLP, PCR).

Conventional plant breeding includes sexual reproduction. Methods may comprise crossing a first parent plant that comprises in its genome at least one copy of a mutation to a second parent corn plant, so as to produce F₁ progeny. The first plant can be any plant or variety including, for example, corn. The second parent plant can be any plant that is capable of producing viable progeny when crossed with the first plant. The first and second parent plants may be of the same corn species (e.g., Zea mays (maize)). Methods may also involve selfing the F₁ progeny to produce F₂ progeny. Methods may further involve one or more generations of backcrossing the F₁ or F₂ progeny plants to a plant of the same line or genotype as either the first or second parent corn plant. Alternatively, the F₁ progeny of the first cross, or any subsequent cross, can be crossed to a third corn plant that is of a different line or genotype than either the first or second plant.

Backcross breeding has been used to transfer genes for simply inherited highly heritable traits into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent until a plant is obtained wherein essentially all of the desired morphological and physiological characteristics of the recurrent parent are recovered in the converted plant, in addition to the transferred locus from the nonrecurrent parent. The resulting plant is expected to have the attributes of the recurrent parent (e.g., cultivar) and the desirable trait transferred from the donor parent. The backcross process may be accelerated by the use of DNA markers (e.g., SSR, RFLP, etc.) to identify plants with the greatest genetic complement from the recurrent parent. In an embodiment, a disclosed process utilizes 1, 2, 3, 4, 5, 6, 7, 8, 9, 10, 15, 20, 25, 30, 40, 45, 50, 60, 70, 75, 90, 100, 110, 120, 125, 150, 170, 175, or 200 DNA markers. Each DNA marker can be separated by 5, 10, 15, 20, 25, or 30 centimorgans (cM). Preferably, each DNA marker is separated by approximately 20 cM.

In embodiments, genomes of the progeny plants are extracted and subjected to LDGA. Once progeny plants have been genotyped, the skilled artisan may select those progeny plants that have a desired genetic composition. Such selected progeny plants may be used in further crosses, selfing, or cultivation.

### Genome Analysis

The linkage drag (LD) can be analyzed by several different methods (e.g., RFLP, PCR, etc.). Described herein, KASPar® PCR assays are discussed. In particular embodiments, a KASPar® PCR assay may be used to assess the linkage drag that occurred following introgression of a desired gene. In embodiments, KASPar® PCR can be utilized in a high throughput system and method for rapidly screening plants and assessing biomarkers in the plant genome.

Primers and probes for use in a gene specific KASPar® PCR assay may be designed based on the gene or allele being introduced. The mutation may be, for example, a single nucleotide polymorphism (SNP) or an insertion or deletion mutation. In some embodiments, the gene specific KASPar® PCR assay may target biomarkers to assess the amount of linkage drag that occurred after introgression.

Target-specific oligonucleotides may be labeled, for example, with fluorescent dyes (e.g., FAM, VIC, and MGBNFQ), which may allow rapid quantification of a target-specific fluorescent signal. PCR products may be measured after a pre-determined number of cycles, for example, when the reaction is in the early exponential phase. Negative control samples may comprise genomic DNA from any plant variety lacking the introgressed gene or allele. Positive control samples may comprise genomic DNA from the parent.

DNA may be isolated (for example, extracted, and purified) from plant tissue by methods known to those of skill in the art. Commercial kits for DNA isolation are available, for example, from Qiagen, Inc. In some embodiments, leaf discs from a particular plant are punched and transferred into collection tubes. The puncher may be cleaned after each sampling with 70% alcohol, rinsing in water, and drying. DNA extraction buffers may be prepared according to the manufacturer's recommendations. DNA may then be isolated using the kit according to the manufacturer's instructions. Finally, the concentration of the isolated DNA may be determined using, for example, a Quant-iT™ PicoGreen® Quantfication Kit (Invitrogen, Carlsbad, Calif.) and a spectrophotometer, or by any other suitable technique.

Once primers, probes, and genomic DNA sample(s) have been prepared or otherwise made available, a competitive allele specific PCR assay (e.g., KASPar®) may be designed using commercial software, such as the Kraken workflow manager, available through KBiosciences (KBiosciences, Hoddesdon, Hertfordshire, UK) to identify nucleic acid sequences of interest in the genomic DNA sample(s). In particular embodiments, individual PCR reaction mixtures are prepared that contain all the reaction components, except the genomic DNA sample(s).

DNA is then amplified by PCR under suitable cycle conditions. In an embodiment, a PCR assay (e.g. KASPar®) is set up with appropriate controls. For example, in some embodiments using a GenAmp® PCR System 9700, there may be a single initial denaturation cycle at 94° C. for 15 minutes, then 20 cycles of denaturation (94° C. for 10 seconds) and annealing (57° C. for 5 seconds) and extension (72° C. for 40 seconds), followed by 22 additional cycles with longer annealing (denaturation at 94° C. for 10 seconds; annealing at 57° C. for 20 seconds, extension at 72° C. for 40 seconds). Those of skill in the art understand that PCR cycle conditions may be varied according to the practitioner's discretion or the specific primer/oligonucleotide sequences involved, and comparable results obtained.

Following completion of a PCR reaction and probe detection, a table and distribution graph may be generated using, for example, any suitable computer graphics software. Raw fluorescence intensity data may also be analyzed directly from a plate reader using a suitable analysis package, such as KLIMS (KBioscience laboratory information management system). A graph with relative fluorescence units (RFU) of a fluorescence signal generated by a specific probe or specific probes can be plotted.

An embodiment includes a method for analyzing plants which contain a desired, introgressed trait comprising analyzing a genome of a plant containing the desired, introgressed trait by determining at least one amount of linkage drag flanking the desired trait on a chromosome containing the desired trait in the genome of the plant; and determining a recurrent parent percentage present on all chromosomes in the genome of the plant, wherein both the determination of the linkage drag and the determination of recurrent parent percentage are performed at a same stage of plant growth. An embodiment further comprises selecting plants containing the desired, introgressed trait based on results of genome analysis.

In a preferred embodiment, linkage drag and genome analysis (LDGA) is conducted as a six step process. First, an intermediate plate of genomic DNA extracted from the progeny is created, where multiple 96 well genomic DNA plates are transferred to a 384 well plate. The 384 well DNA plate serves as the DNA source plate for all PCR reactions. Then the genomic DNA is dispensed to PCR plates (e.g., 1536 well plates). Then the DNA is dried by methods well known in the art. DNA can be dried for 30, 60, 90, 120, 240, 270, 300, 330, or 360 minutes on PCR plates. In a preferred embodiment, DNA can be dried on PCR plates for 120 minutes. After drying, a PCR reaction mixture (or “CKTL”) is dispensed to each well of a PCR plate. The plates then undergo thermal cycling under suitable conditions. After the PCR reaction is complete, the fluorescent probes are detected and/or quantified by fluorescent plate readers. Data are collected and can be analyzed by various software applications.

### EXAMPLES

### Example 1

### Original Process, Linkage Drag followed by Genome Analysis

The purpose of a linkage drag (LD) project is to remove linkage drag (the part of the chromosome that is not homozygous for the elite parent). The original method of analyzing introgression of a desired trait into a plant genome utilized a LD project followed by genome analysis (GA). The genomic DNA for a project was originally extracted in a 96 well format, which was then transferred into an intermediate 384-well plate. The intermediate plate served as a diluted source plate which was then stamped into a known number of 1536 or 384 well PCR reaction plates, depending on how many samples and markers were needed for the project. Typically, 186 samples were sent in for extraction, and markers were chosen every 10 cM on the chromosome(s) with the insert region. Following linkage drag analysis, a genome analysis project was performed, which focused on finding the top 12 plants with the largest amount of Recurrent Parent Percentage (RPP) while balancing the amount of LD. With GA projects, the top 45 samples were selected from the 186 samples sent in for the LD project and analyzed across all chromosomes (except the chromosome that was analyzed using the LD project) at a distance of 20 cM between markers. This process is performed as two separate projects, not simultaneously. The LD (n=186) was set up in a 1.3 μl total reaction per well across 1536 well plates, and the GA (n=45) was set up in a 5 μl total reaction in 384 well plates.

The original process, LD followed by GA, includes 13 steps as described as follows:
1. For linkage drag analysis, create an intermediate plate by transferring DNA from 96 wells plates to a 384 well plate.
2. Dispense the DNA from the 384 well intermediate plate to a 1536 PCR plate.
3. Dry down the DNA for 2 hours in a 65° C. oven.
4. Dispense the PCR cocktail (CKTL) to the PCR plates.
5. Thermal cycle PCR plates.
6. Read the resulting fluorescent signal with a plate reader.
7. Hit-pick the top 45 plants or samples for genome analysis.
8. For genome analysis, another intermediate plate is created by transferring DNA from 96 wells plates to a 384 well plate.
9. Dispense the DNA from the 384 well intermediate plate to a 1536 PCR plate.
10. Dry down the DNA for 2 hours in a 65° C. oven.
11. Dispense the PCR cocktail (CKTL) to the PCR plates.
12. Thermal cycle PCR plates.
13. Read the resulting fluorescent signal with a plate reader.

### Example 2

### Improved Process, Simultaneous LDGA

A new system was established to eliminate performing the LD+GA analysis as two separate projects. The simultaneous LDGA process was performed in 1536 well plates, allowing for a reduced total reaction to 1.3 μl per well across the plate, reduced consumable costs, reduced setup time per project, and enhanced high throughput capabilities. This improved process is capable of producing more data in shorter timeframes. The simultaneous LDGA process includes steps 1 through 6, as shown in Example 1, and eliminates 7 steps, from hit-picking the top 45 plants through the second fluorescent plate reading step (steps 7 through 13, shown in Example 1).

### Example 3

### Cost Analysis, Original Process Versus Improved Process

Cost analysis was performed on average project sizes for both LD and GA analysis. The cost was calculated for the KBioscience Competitive Allele-Specific PCR genotyping system, or KASPar™, (KBiosciences, Hoddesdon, Hertfordshire, UK), based on the manufacturer's protocols. The cost per data point was calculated for each component of the PCR cocktail and for all consumables used to set up the PCR reactions. Tables 1 and 2 detail the cost analysis for the LD and GA analysis, respectively, for the original process. Cost for the improved process is the same as the LD analysis for the original, which is shown in Table 1. Performing LD and GA as two separate projects resulted in a cost of $0.20 per data point, while performing LD and GA simultaneously resulted in a cost of $0.04 per data point. A savings of $0.16 per data point was realized with the improved process. Significant savings is realized with larger sample sizes and increased marker numbers.

TABLE 1
Cost analysis for the LD portion of the original process, which
is the same for the improved, simultaneous LDGA process. Cost
is measured per data point based on a 1536 well PCR plate.

| Component | Cost per unit | Cost per Data Point |
|---|---|---|
| Master Mix | $16.68/ml | $0.01 |
| Primers | $0.01/reaction | $0.01 |
| 1536 PCR plate | $11.00/plate | $0.01 |
| 384 well intermediate plate | $3.50/plate | $0.01 |
| Total | | $0.04 |

TABLE 2
Cost analysis for the GA portion of the original process. Cost
is measured per data point based on a 384 well PCR plate.

| Component | Cost per unit | Cost per Data Point |
|---|---|---|
| Master Mix | $14.50/ml | $0.04 |
| Primers | $0.01/reaction | $0.01 |
| 384 PCR plate | $3.10/plate | $0.01 |
| 384 well intermediate plate | $3.50/plate | $0.01 |
| 96 well plate for hit-picking | $3.20/plate | $0.03 |
| tips for hit-picking | $5.70/rack | $0.06 |
| Total | | $0.16 |

### Example 4

### Time Analysis, Original Process Versus Improved Process

In addition to being more cost effective, the simultaneous LDGA process reduced overall project time in the lab and in the field. By reducing the number of projects from two projects to one, set up time in the lab was reduced, hit-picking samples was eliminated, and time for data analysis was also reduced. In the field, overall time researchers spent pollinating was reduced. The original process identified 45 top plants for pollination from the LD analysis, while the newly combined process identified only 12 top plants for pollination from the LDGA analysis. Table 3 summarizes the time saving opportunities identified with the improved process. Table 4 summarizes the average time savings realized with the improved LDGA process, which totals 7 hours and 2 minutes.

TABLE 3
Time saving opportunities available with
the improved, simultaneous LDGA process.

| Task | Original LD then GA | Improved LDGA |
|---|---|---|
| Lab | set up for 2 separate projects (LD on 1536 | lab set up for 1 |
| | then GA on 384) | project (LDGA on |
| | | 1536) |
| | data analysis for 2 separate projects (LD | data analysis for 1 |
| | then GA) | project (LDGA) |
| | hit-pick top 45 samples from LD analysis | no hit-picking |
| Field | pollinate top 45 plants from LD analysis | pollinate top 12 plants |
| | | from LD analysis |
| | LD data received by pollination; GA data | LDGA data received |
| | received by harvest | by pollination |

TABLE 4
Average time savings per project with the
improved, simultaneous LDGA process.

| Task | Original LD then GA | Improved LDGA |
|---|---|---|
| Set-up and Analysis | 7 hours 28 minutes | 3 hours 44 minutes |
| Lab time | 4 hours 30 minutes | 3 hours |
| Hit-picking | 42 minutes | n/a |
| Field Time | 1 hour 30 minutes | 24 minutes |
| Total time | 14 hours 10 minutes | 7 hours 8 minutes |

1. A method for analyzing plants which contain a desired, introgressed trait, the method comprising the steps of: analyzing a genome of a plant containing the desired, introgressed trait by (i) determining at least one amount of linkage drag flanking the desired trait on a chromosome containing the desired trait in the genome of the plant; and, (ii) determining a recurrent parent percentage present on all chromosomes in the genome of the plant, wherein both the determination of the linkage drag and the determination of recurrent parent percentage are performed at a same stage of plant growth; and selecting plants containing the desired, introgressed trait based on results of genome analysis.

2. The method of claim 1, wherein the analysis utilizes 10, 20, 25, 30, 40, 45, 50, 60, 70, 75, 90, 100, 110, 120, 125, 150, 170, 175, or 200 DNA markers.

3. The method of claim 2 wherein the DNA markers are about 20 cM apart.

4. The method of any one of claims 1 to 3, wherein the analysis of the genome is based on high throughput screening.

5. The method of claim 4, wherein the high throughput screening comprises conducting PCR on a plurality of plant genomes.

6. The method of claim 5, wherein the plurality of plant genomes is greater than 186 genomes.

7. The method of claim 5, wherein the plurality of plant genomes is greater than 1500 genomes.

8. The method of claim 5, wherein the PCR is single nucleotide polymorphism genotyping.

9. The method of claim 8, wherein the genomes are contacted with three primers, wherein two primers are allele specific and one primer is a common reverse primer.

10. The method of claim 5, wherein the PCR is fluorescent multiplex PCR.

11. The method of claim 1 further comprising contacting the plant genome with PCR primers specific for a marker linked to the introgressed trait.

12. The method of any one of claims 1 to 11 further comprising selecting the top 5, 10, 12, or 15 samples from the genome analysis.

13. A method of genome analysis comprising screening a genome by linkage drag genome analysis (LDGA) genotyping, wherein only one round of PCR is performed.

14. The method of claim 13, further comprising delivering LDGA genotyping results solely by flowering.

15. A method of genome analysis comprising delivering linkage drag genome analysis (LDGA) genotyping results solely by flowering.

16. The method of according to any one of claims 13 to 15, wherein the genome is a plant genome.

17. The method of according to any one of claims 13 to 16, wherein the genome is a crop genome.

18. The method of according to claim 17, wherein the crop genome is maize.

19. The method according to claim 17, wherein the crop genome is a monocot.

20. The method according to claim 19, wherein the monocot is barley, wheat, rice, oat, rye, sugarcane, or sorghum.

21. The method according to claim 17, wherein the crop genome is a dicot.

22. The method according to claim 21, wherein the dicot is tobacco, soybean, sunflower, peanut, cotton, or tomato.

23. The method according to claim 21, wherein the dicot is a legume.

24. The method according to any one of claims 13 to 23, wherein the screening uses a multi-well configuration with at least 1500 separate wells.

25. The method according to claim 24, wherein the multi-well configuration is a 1536 well configuration.

26. The method according to any one of claims 13 to 25, wherein the method is free of hit-picking.

27. The method according to any one of claims 13 to 26, wherein the genotyping utilizes a homogeneous fluorescent resonance energy transfer (FRET) based system.
